(19) United States
(12) Patent Application Publication
(10) Pub. No.: US 2013/0296939 A1
(43) Pub. Date: Nov. 7, 2013

(54) ADJUSTABLE SPINOUS PROCESS SPACER DEVICE AND METHOD OF TREATING SPINAL DISORDERS

(71) Applicant: Richard PERKINS, Staatsburg, NY (US)

(72) Inventor: Richard PERKINS, Staatsburg, NY (US)

(21) Appl. No.: 13/209,427

(22) Filed: Aug. 14, 2011

Related U.S. Application Data
(63) Continuation-in-part of application No. 11/600,321, filed on Nov. 16, 2006, now Pat. No. 7,998,173.
(60) Provisional application No. 60/738,930, filed on Nov. 22, 2005.

(51) Int. Cl. A61B 17/70

(52) U.S. Cl. CPC A61B 17/7068
USPC 606/249; 606/279

(57) ABSTRACT

Provided is a bi-dimensionally adjustable spacing device configured to be placed between the spinous processes of at least two adjacent vertebrae and also adjusted laterally to securely contact the lateral surfaces of the spinous process. A method of using the device to treat spinal disorders is also provided.

The present application is a continuation in part application of U.S. patent application Ser. No. 11/600,321, filed Nov. 16, 2006, which is a non-provisional application of U.S. provisional application Ser. No. 60/738,930, filed Nov. 22, 2005, the complete disclosure of both are herein incorporated by reference.

### BACKGROUND

1. Technical Field

The present invention relates to devices and methods for use in spine surgery. In particular, the present invention relates to a bi-dimensionally adjustable spacing device configured to be placed between the spinous processes of at least two adjacent vertebrae and also adjusted laterally to securely contact the lateral surfaces of the spinous process. A method of using the device to treat spinal disorders is also provided.

2. Background Art

The human spine is comprised of thirty-three vertebrae at birth and twenty-four as a mature adult. Between each pair of vertebrae is an intervertebral disc, which maintains the space between adjacent vertebrae and acts as a cushion under compressive, bending and rotational loads and motions. A healthy intervertebral disc has a great deal of water in the well hydrated nucleus pulposus, the center portion of the disc. The water content gives the nucleus a spongy quality and allows it to absorb spinal stress.

In a young healthy individual, the intervertebral disc also serves as a natural spacer between adjacent vertebrae thus allowing sufficient space in the central spinal canal and intervertebral foramina to permit unimpeded nerve passage from the neural canal. In addition to injuries or disease of the intervertebral discs, the common condition of spinal stenosis can impinge upon neural and vascular structures leading to neurological compromise.

Spinal stenosis is a common condition resulting from the narrowing of the spinal canal, nerve root canals and intervertebral foramina causing nerve pinching, which leads to persistent pain, lack of feeling and decreased physical activity. The narrowing of nerve egress pathways from the vertebral column results in compression of spinal nerves and nerve roots, causing a constellation of symptoms, including lower back pain, neurogenic claudication and lower extremity pain.

While spinal stenosis is a pervasive cause of such pain and disability, there are many other spinal conditions, diseases, and injuries among patients of all ages that create a need to provide effective relief for a growing population of spinal disorder patients.

### SUMMARY OF THE DISCLOSURE

Disclosed herein is a device that meets the above identified need by providing a novel device and method for the treatment of spinal disorders. More particularly, a device is disclosed that provides a spacer, adjustable on two planes that can be easily positioned between adjacent spinous processes so as to relieve the pressure between adjacent vertebrae as a treatment for spinal disorders.

Disclosed is a device for use in the treatment of spinal disorders that includes an assembly having two approximately parallel elongated legs, the legs being adjustably connected one to the other by transverse members that are adjustable in two planes.

Also disclosed is a device for treating spinal disorder and injuries that includes an assembly having two approximately parallel elongated legs being adjustably connected one to the other by transverse members that are adjustable in two planes with each transverse member having a shape suitable to abut against a respective spinous process of a vertebrae so as to form a device having a box-like configuration abutted against and between at least one pair of adjacent spinous processes of respective adjacent vertebrae.

Also disclosed in as method of treating spinal stenosis that includes providing the device disclosed herein and using surgical methods to position the device on the dorsal side of the spinal column of a patient such that the two legs are positioned one on either side of the center of the spinal column and approximately parallel thereto, positioning the first transverse member against the spinous process of a first vertebra and moving the adjacent adjustable transverse member to a position abutting against the spinos process of a second adjacent vertebra and adjusting the relationship of the transverse member to each of the two legs disposed on each side of the spinous processes, locking the adjustable transverse member into position on the two legs so as to form an adjustably fixed spacer between the first spinous process and the second adjacent spinous process.

### BRIEF DESCRIPTION OF THE DRAWINGS

The foregoing and other features of the present invention will become apparent to one skilled in the art to which the present invention relates upon consideration of the following description of the invention with reference to the accompanying drawings, wherein:

FIG. 1A shows a dorsal view of two adjacent vertebrae having a first exemplary embodiment of the bi-dimensionally adjustable spinous process spacer device inserted between and abutted against adjacent spinous processes with the first and second elongated, longitudinal legs positioned away from each other and not in contact with the spinous process located there between.

FIG. 1B shows a dorsal view of two adjacent vertebrae having a first exemplary embodiment of the bi-dimensionally adjustable spinous process spacer device inserted between and abutted against adjacent spinous processes with the first and second elongated, longitudinal legs positioned toward each other and in contact with the spinous process located there between.

FIG. 2 shows a side view of a first and a second adjacent vertebra having a first exemplary embodiment of the bi dimensionally adjustable spinous process spacer device inserted between the two adjacent spinous processes, the device having a caudally inclining transverse member abutted against the first spinous process and a cephalid inclining transverse member abutted against the second spinous process so as to hold the two adjacent vertebra in a fixed spatial relationship one to the other. Also shown are gradation indicia on the surface of the longitudinal legs.

FIG. 3 shows a front view of a transverse member of a first exemplary embodiment of the bi-dimensionally adjustable spinous process spacer device. On a first end of the transverse member is shown an exemplary locking device for the elongated, longitudinal legs, which as seen from the end are positioned within their respective oblong through portals in the transverse member, the first exemplary locking device, which is shown on the right includes a threaded grommet having an expansion cut, the grommet being slidably engaged within an elongated locking portal and a set screw capable of threadably engaging the threaded slidable grommet so as to laterally expanded the expansion cut of the grommet pressing the grommet against the walls of the elongated locking portal to lock its relative position within the locking portal and to securely engage and hold in position the elongated, longitudinal leg. A second exemplary locking device is shown in a pre-insertion position on the left side of the depiction of a transverse member, the exemplary locking device being an outwardly biased insertable clip having a locking notch to engage and exert an outward holding pressure against the elongated legs within the oblong through portal defined in the transverse member.

FIG. 4 shows a detail of the threaded slidable grommet of the first exemplary embodiment shown in FIG. 3 positioned within the elongated locking portal defined in the upper portion of the end of the transverse member.

FIG. 5 shows a frontal perspective view of a second exemplary embodiment of the bi-dimensionally adjustable spinous process spacer device, the embodiment having an upper and a lower transverse member defined through said longitudinal members, the device employing a collet locking device in the upper slot in the longitudinal plane and lateral plane of the device and using a set screw locking device for securing a transverse member in the lower slot in a selected position in the lateral plane, the collet mechanism and set screw locking in place a transverse member in the upper slot and a transverse member in the lower slot and holding the adjacent spinous process in a fixed relationship one to the other.

FIG. 6 shows an exploded view of the second exemplary embodiment of the bi-dimensionally adjustable spinous process spacer device of FIG. 6 having a collet lock.

FIG. 7 shows the second embodiment of the bi-dimensionally adjustable spinous process spacer device of FIG. 6 having a collet lock in position between two adjacent vertebrae, holding the two adjacent spinous processes in a fixed spatial relationship one to the other and having the device securely abutted against the lateral surfaces of the spinous process.

FIG. 8 shows a frontal perspective view of a third exemplary embodiment of the bi-dimensionally adjustable spinous process spacer device having a fitted lateral ratchet locking device to secure the device between two adjacent vertebra and holding the adjacent spinous process in a fixed spatial relationship one to the other while employing set screws to secure the device in a selected position on either side of the spinous process of a vertebra.

FIG. 9 shows an exploded view of the third exemplary embodiment of the bi-dimensionally adjustable spinous process spacer device of FIG. 6 having a fitted lateral ratchet locking device.

FIG. 10 shows three devices of the third exemplary embodiment of the bi-dimensionally adjustable spinous process spacer device of FIG. 6 having a fitted lateral ratchet locking device in position between adjacent vertebrae, holding the adjacent spinous processes in a fixed spatial relationship one to the other and having the device securely abutted against the lateral surfaces of the respective spinous processes.

FIG. 11 shows an alternative configuration of the fitted lateral ratchet embodiment shown in FIGS. 8-10 of the bi-dimensionally adjustable spinous process spacer device using a set screw for securing the fitted ratchet in place relative to the transverse member. Also shown is the device having multiple spike contacts to augment the security of the device against the lateral surface of the spinous process.

FIG. 12 shows a frontal view of a fourth exemplary embodiment of the bi-dimensionally adjustable spinous process spacer device having a rotating gear mechanism for adjusting the distance between transverse members positioned between the spinous processes of adjacent vertebra. Also shown is the set screw locking device for securing the lateral position plates equipped with spike fixation augmentation members against the lateral surfaces of the spinous process.

FIG. 13 shows a posterior perspective view of the fourth exemplary embodiment of the bi-dimensionally adjustable spinous process spacer device shown in FIG. 12.

FIG. 14 shows an exploded view of the fourth exemplary embodiment of the bi-dimensionally adjustable spinous process spacer device shown in FIG. 12.

FIG. 15 shows the rotating vertically oriented gear for adjusting the distance between transverse members positioned between the spinous processes of adjacent vertebra of the fourth exemplary embodiment shown in FIG. 12.

FIG. 16 shows a frontal perspective view of a fifth exemplary embodiment of the bi-dimensionally adjustable spinous process spacer device having a fitted posterior ratchet locking device to secure the device in a selected spatial relationship between the spinous processes of two adjacent vertebrae and using set screws to secure the device in a selected position against the lateral surface of the spinous process.

FIG. 17 shows an exploded view of the fifth exemplary embodiment of the bi-dimensionally adjustable spinous process spacer device of FIG. 16 having a fitted posterior ratchet locking device.

FIG. 18 shows two devices of the fifth exemplary embodiment of the bi-dimensionally adjustable spinous process spacer device of FIG. 16 having a fitted posterior ratchet locking device in position between adjacent vertebrae, holding the adjacent spinous processes in a fixed spatial relationship one to the other and having the device securely abutted against the lateral surfaces of the respective spinous processes.

FIG. 19 shows a sixth exemplary embodiment of the bi-dimensionally adjustable spinous process spacer device with two adjacent transverse members mounted on substantially parallel elongated members, the elongated members having a plurality of gripping devices such as, for example, a toothed or ratcheted surface and being securable to the transverse members at selected positions by set screw locking blocks, the locking blocks also including spinous process lateral surface contact security elements such as, for example, inwardly directed spikes.

FIG. 20 shows an exploded view of the sixth exemplary embodiment of the bi-dimensionally adjustable spinous process spacer device shown in FIG. 19.

FIG. 21 shows two devices of the sixth exemplary embodiment of the bi-dimensionally adjustable spinous process spacer device of FIG. 19 positioned on adjacent vertebrae, the caudally position device being adjusted inwardly so as to bring the spinous process contact security elements into contact with the lateral surfaces of the spinous process and the superior positioned device not yet fully adjusted inwardly to cause contact between the contact security elements and the lateral surface of the spinous process of the superior vertebra.

### DETAILED DESCRIPTION OF PREFERRED EMBODIMENTS

Detailed embodiments of the present invention are disclosed herein; however, it is understood that the following description and each of the accompanying figures are provided as being exemplary of the invention, which may be embodied in various forms without departing from the scope of the claimed invention. Thus, the specific structural and functional details provided in the following description are non-limiting, but serve merely as a basis for the invention as defined by the claims provided herewith. The device described below can be modified as needed to conform to further development and improvement of materials without departing from the inventor's concept of the invention as claimed.

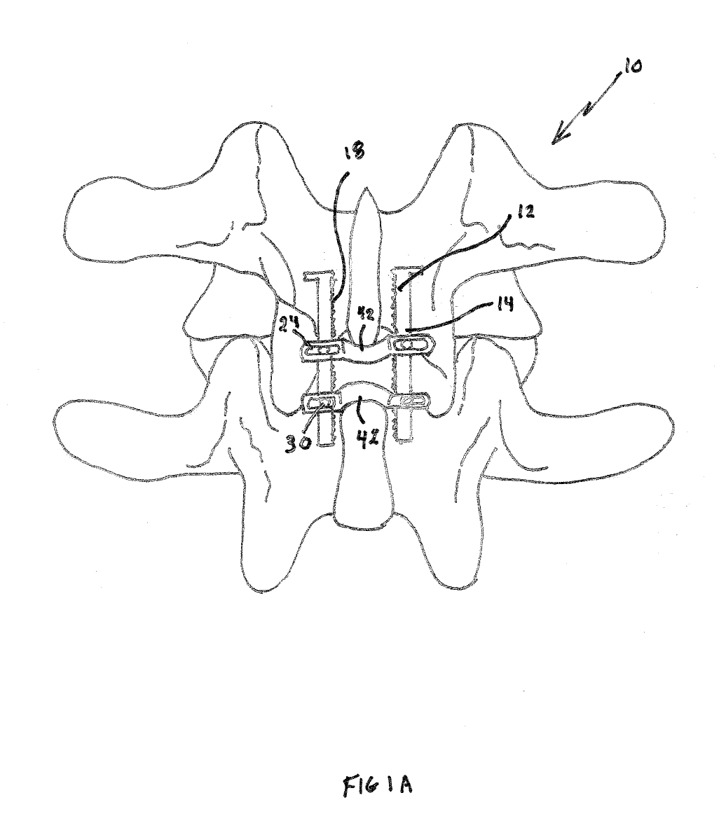

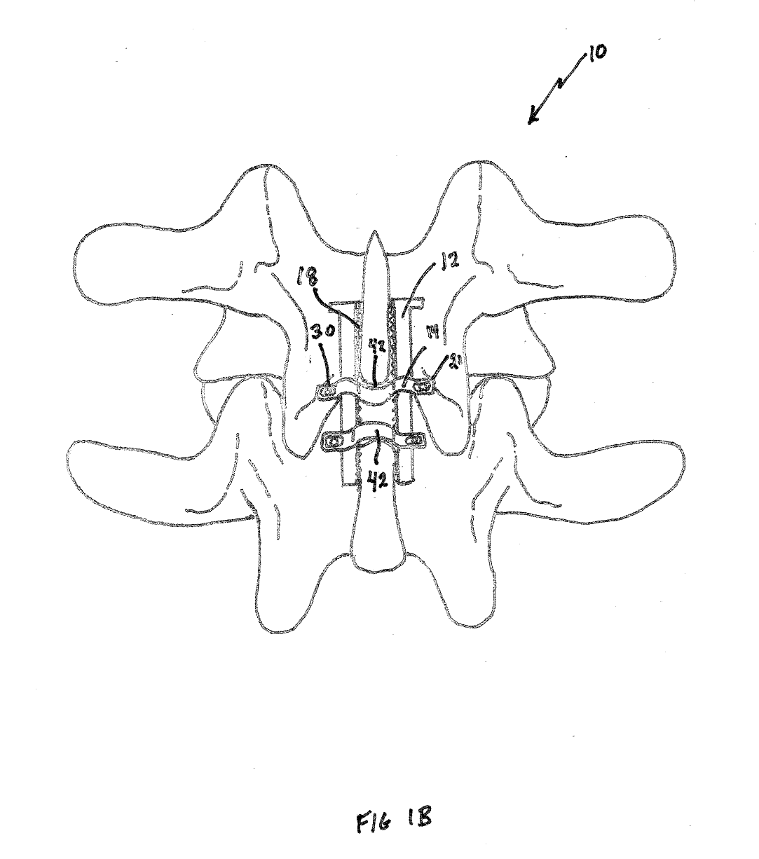

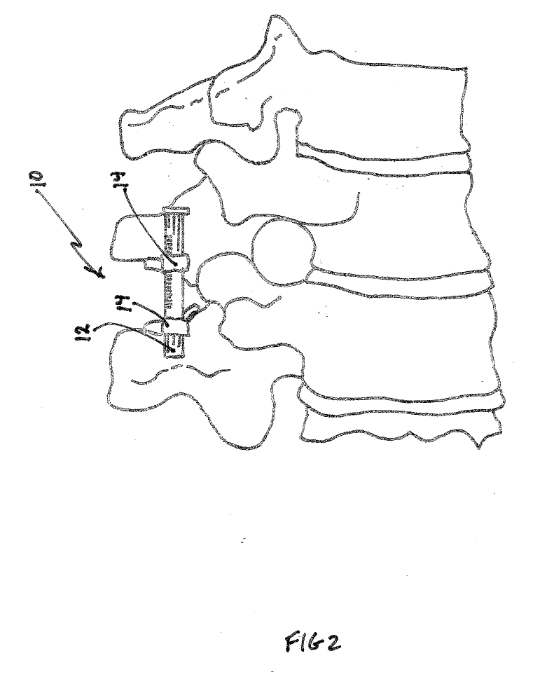

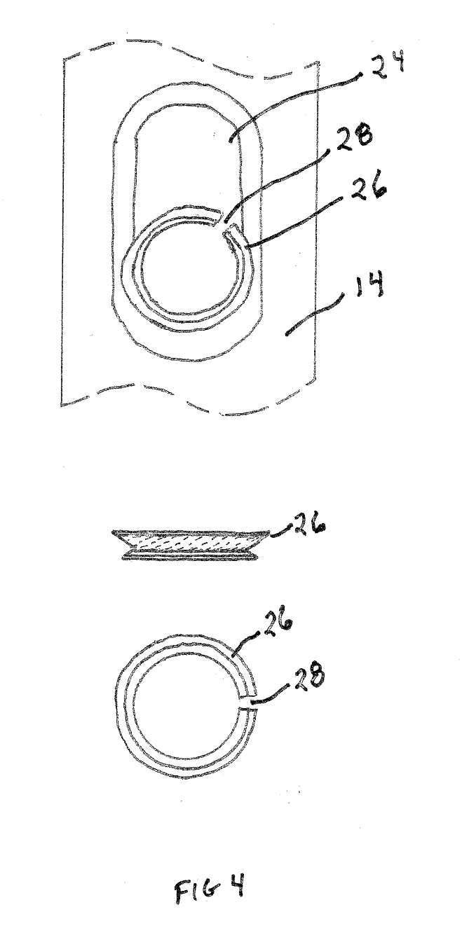

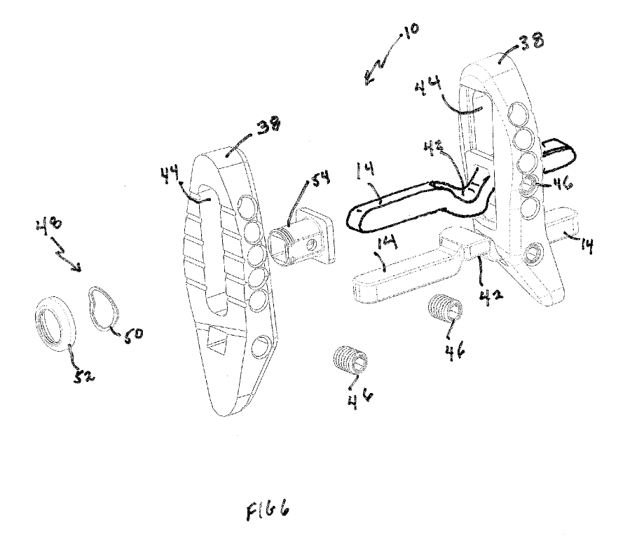

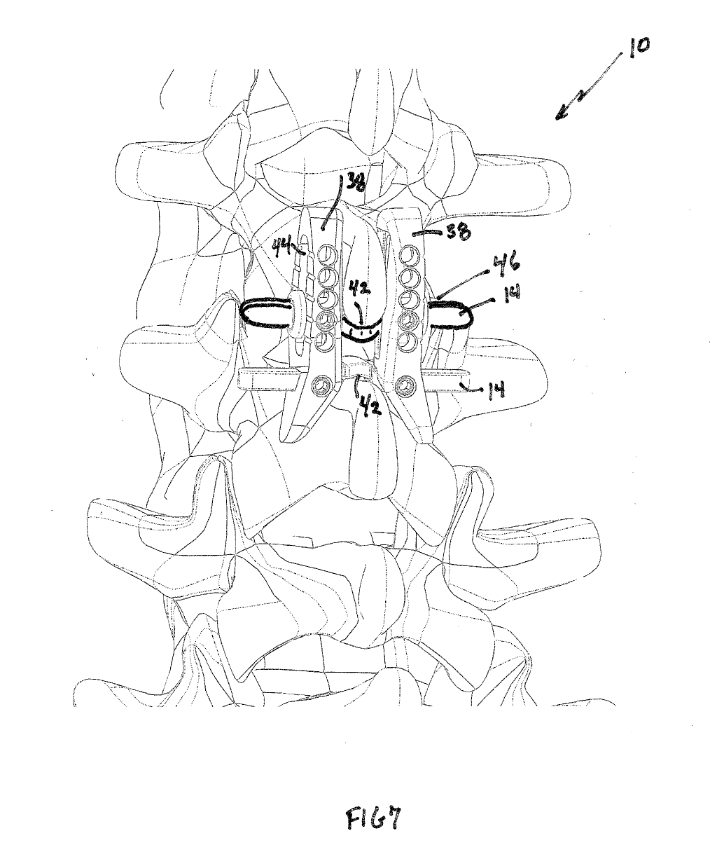

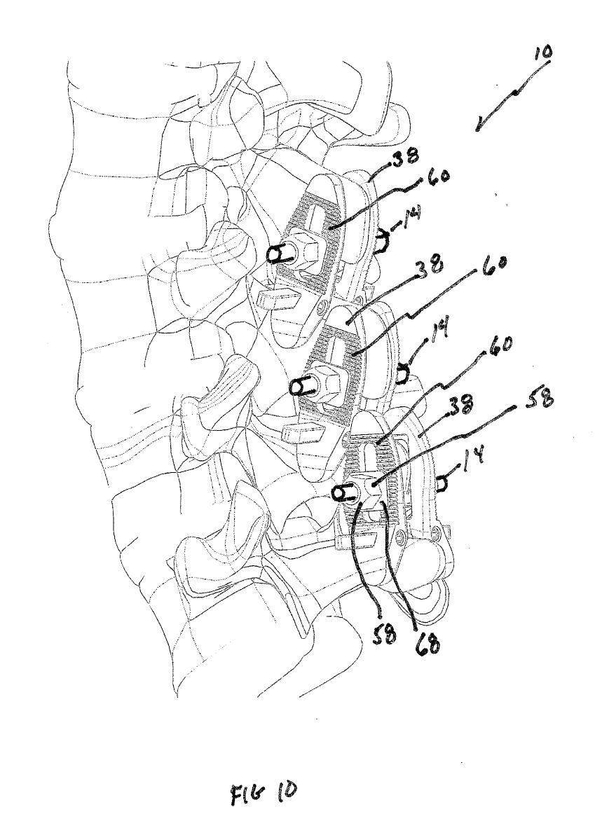

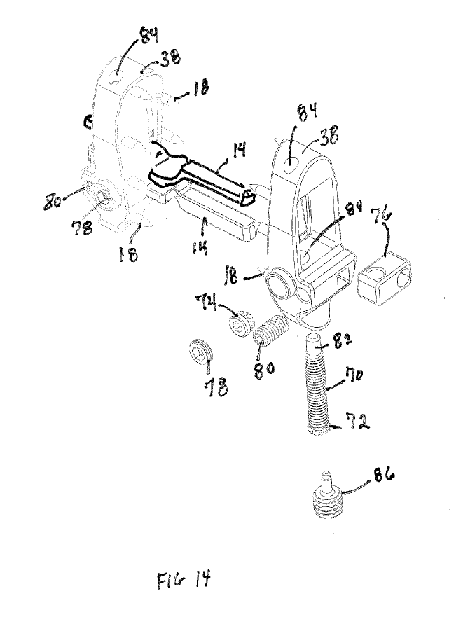

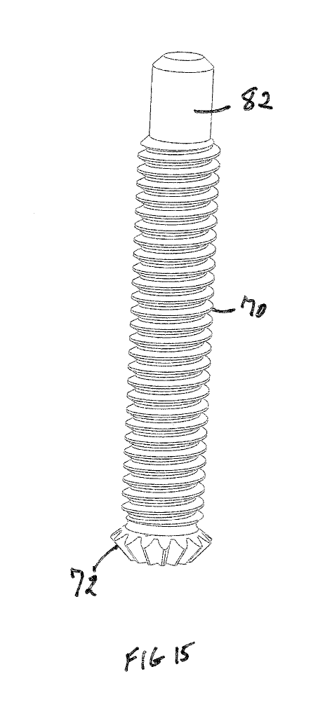

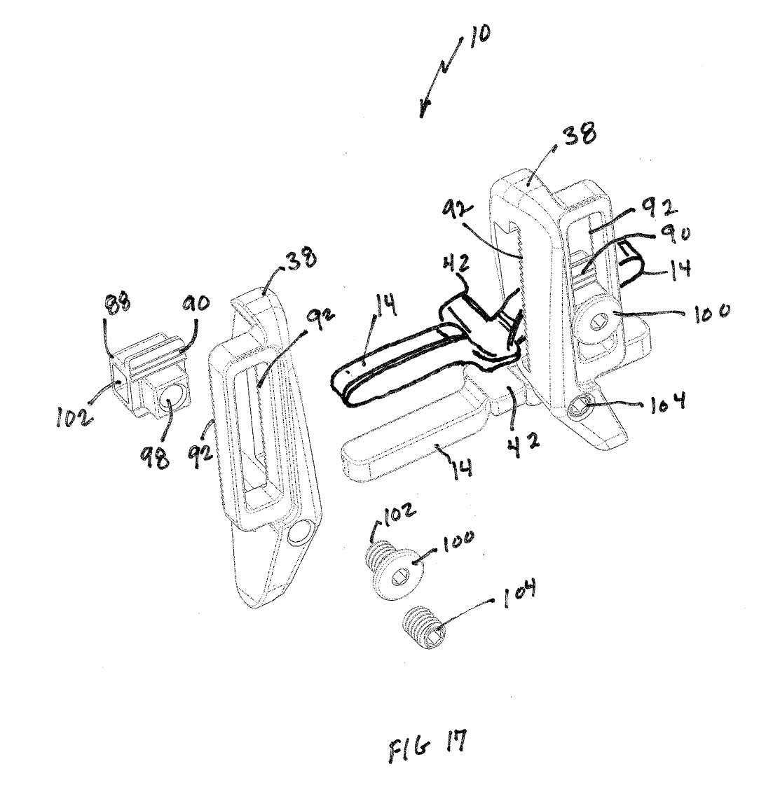

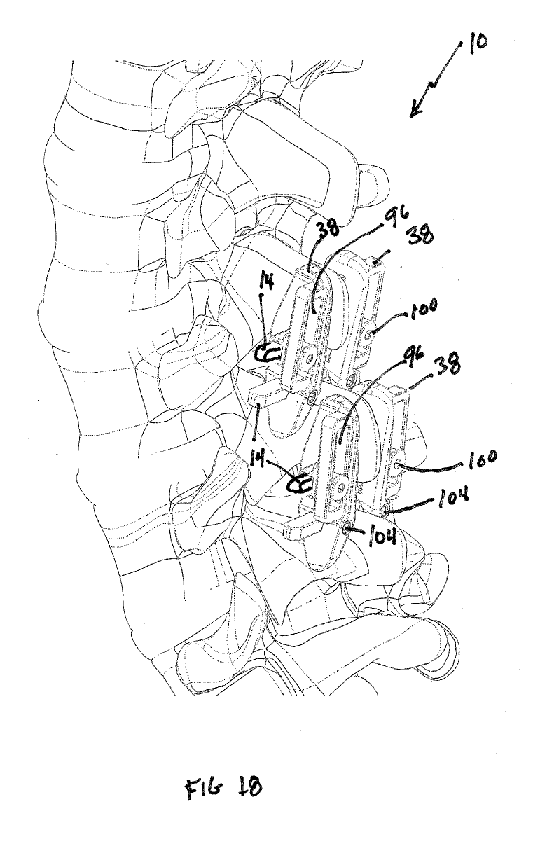

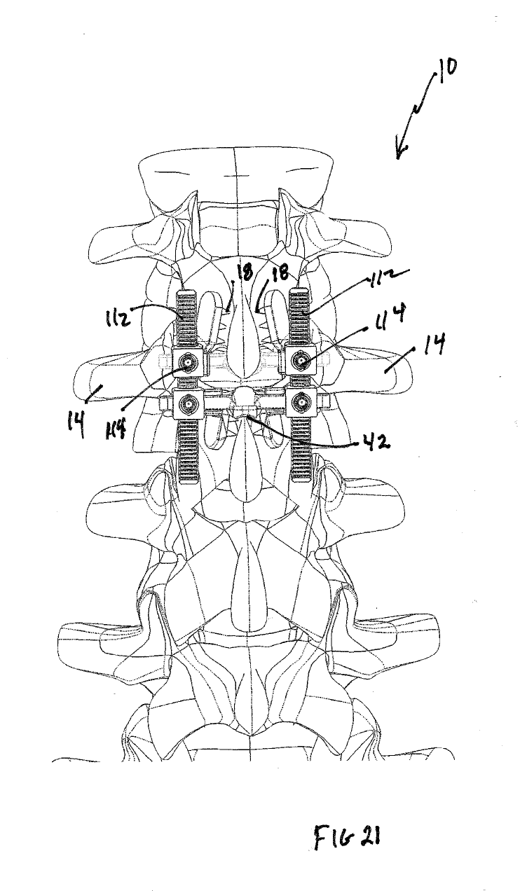

The device and components thereof, as generally shown at 10 in FIG. 1 through FIG. 21, is a novel bi-dimensionally adjustable spinous process spacer device capable of securely maintaining a selected distance between the spinous processes of two or more adjacent vertebrae and also allowing for lateral adjustment and selected positioning of elements of the device 10 so as to make secure contact with the lateral surfaces of the spinous process of the vertebrae to which the device is attached.

As demonstrated in the discussions of each of the exemplary embodiments of the device 10, the general concept of providing a device 10 that is capable of being adjusted relative to the spinal vertebrae on two planes, longitudinal adjustment and lateral adjustment, and securely locked into place is constant with the only variations being the selected mechanism for accomplishing the function of the inventive concept. Another common feature of each of the exemplary embodiments discussed below is the capability for releasably securing the transverse member 14 at selected positions relative to the first or second longitudinal members 12 shaped and configured as elongated legs or as side plates 38 of the device.

As shown in the first exemplary embodiment of the bi-dimensionally adjustable spinous process spacer device, shown in FIGS. 1A through FIG. 4, two relatively parallel longitudinal members 12, configured as elongated legs, can be positioned along the longitudinal axis of the spine and on either side of the spinous processes of the underlying vertebrae. Transverse members 14 can be provided having oblong through portals 16 sized and configured to allow slidable passage of the longitudinal members as configured in this embodiment as elongated legs 12. The oblong through portals 16 permit longitudinally directed movement of the transverse members 14 relative to the spinous process of adjacent vertebrae and relative to other transverse members 14. Additionally, the oblong through portals 16 allow the elongated legs 12 to be adjusted laterally so as to bring the transverse members into a position in contact with the lateral surface of the spinous process. FIGS. 1A and 1B best demonstrate this lateral positioning of the elongated legs 12 and the transverse members 14 relative to the spinous process of the vertebra. As shown in FIG. 1A, the elongated legs are extended within the oblong through portals 16 laterally, away from the spinous process of the vertebra. As shown in FIG. 1B, the elongated legs 12 have been laterally adjusted inward within the oblong through portals 16 so as to bring the elongated legs 12 into contact with the lateral surface of the spinous process. Surface contact structures 18, which can be in the form of a textured surface, ribs, spikes, or any other frictional contact augmenting structures can also be provided on the surface of the elongated legs 12 for the purpose of improving secure contact between the elongated legs 12 and the lateral surface of the spinous process.

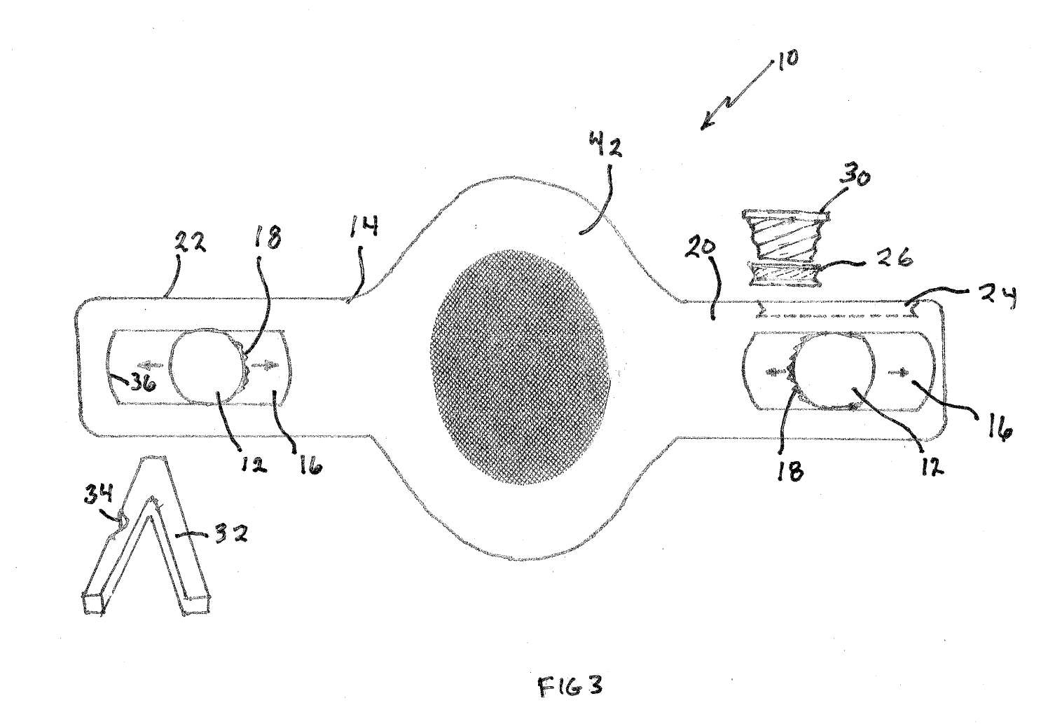

As shown in FIGS. 1A, 1B, and FIG. 3, this first exemplary embodiment of the device 10, can be secured into a selected configuration longitudinally relative to the spinous processes of adjacent vertebra and laterally relative to the lateral surfaces of the spinous processes by more than one locking system. Two exemplary locking devices for the first described embodiment of the device 10 are shown in FIG. 3. These non-limiting examples of locking devices demonstrate a wide variance in locking devices that can be adapted to this embodiment of the device 10. As shown on the right side 20, the upper surface of the lateral extensions of the transverse member 14 can define an elongated locking portal 24 located directly over the oblong through portal 16 through which the elongated legs 12 pass. The oblong through portal 16 can be provided with a threaded grommet 26 configured to be slidably engaged and adjustably held within the through portal 16. Provision of an expansion cut 28 facilitates inward compression of the threaded grommet for assembly within the through portal 16. The provision of the expansion cut 28 is also advantageous when a locking screw 30 is threaded into the threaded grommet 26 and due to the oversized dimension of the locking screw 30 effects an outward expansion of the threaded grommet 26 thus locking the screw 30, the threaded grommet 26 and the longitudinal leg 12 into a secure, locked position relative to each other and the vertebra and the spinous process.

An alternative locking device to secure the longitudinal legs into a fixed position within the oblong through portals in the transverse members 14 is shown on the left side 22 of the transverse member 12 shown in FIG. 3 in a pre-insertion position relative to the transverse member 14. The exemplary locking device as shown is an outwardly biased insertable clip 32 having a locking notch 34 to engage and exert an outward holding pressure against the elongated legs within the oblong through portal defined in the transverse member. When the clip 32 is fully inserted, the locking notch 34 engages the outer edge 36 of the oblong through portal 16 and securely holds the elongated leg 12 in its selected position relative to the transverse member 14 and the spinous process of the adjacent vertebra.

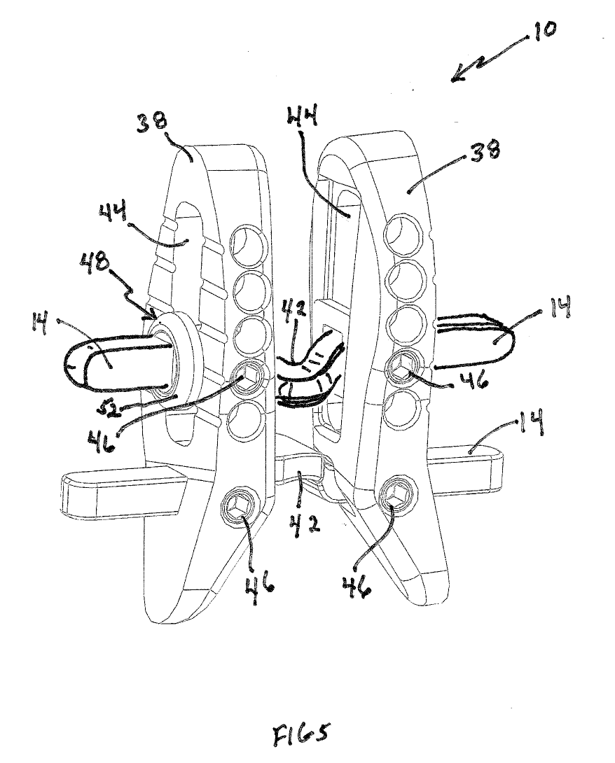

A second exemplary embodiment of the device 10 as shown in FIGS. 5-7 is, as other embodiments, capable of being adjusted longitudinally so as to set and hold the relative position of two adjacent spinous processes while also permitting adjustment of the transverse members relative to the elongated legs 12 so as to bring the outlying elongated legs into a secure contact position against the lateral surfaces of the spinous process. As shown in FIGS. 5-7, rather than providing the first and second longitudinal members 12 configured as elongated legs 12 as in the first exemplary embodiment, they can also be configured as parallel oriented side plates 38 as shown in some of the other exemplary embodiment. When the longitudinal members are provided as a broader shaped side plate 38 configuration, the transverse members 14 pass through upper and lower transverse member adjustment slots 44 and 45 defined through the side plates 38. The upper transverse member adjustment slot 44 being elongated along the longitudinal axis of the side plate 38, permits adjustment of the transverse members 14 in both the longitudinal and lateral planes of the device. The transverse member adjustment slot 45 being relatively the same shape as the cross-section of the lower transverse member 45, allows adjustment of the side plate 38 relative to the transverse member only in the lateral plane of the device. The transverse members 14, as in other exemplary embodiments described herein, are provided with a first end, a second end, and a central portion connecting the first end to the second end, the central portion being ergonomically configured to be curved in a caudal or cephalic direction along substantially the length of the central portion, that curve being away from the longitudinal axis of the transverse member 14 so as to abut against and hold in position a respective spinous process of adjacent vertebra. This sloping configuration of the central portion of the transverse members 14 provides a contact surface for the device 10 that is better conformed to the shape of the spinous processes with which the central portion 42 of the transverse member 14 makes contact. As shown in FIGS. 5-7 the adjustment of the transverse members 14 upward or downward along the longitudinal axis of the side plates 38 is accomplished by adjustment of the transverse member end portions within the elongated transverse member adjustment slots 44 defined laterally through the body of the side plates 38. When the transverse members 14 are selectively adjusted to the desired location within the adjustment slots 44, their relative positions can be securely locked using a set locking set screw 46. Additionally, a collet lock mechanism, generally shown at 48 can be provided as shown in FIGS. 5-7. the tension member 50, the locking cap 52, and the transverse member guide 54 each being configured to permit the lateral end of the transverse member to pass through serve to guide the transverse member upward or downward within the transverse member adjustment slots 44 and exert a holding tension on the end portions of the transverse member 14 keeping it in a secure track position within the side plates 38.

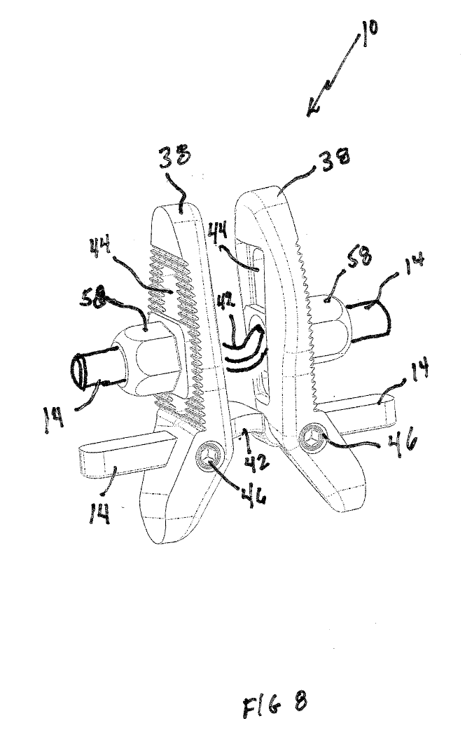

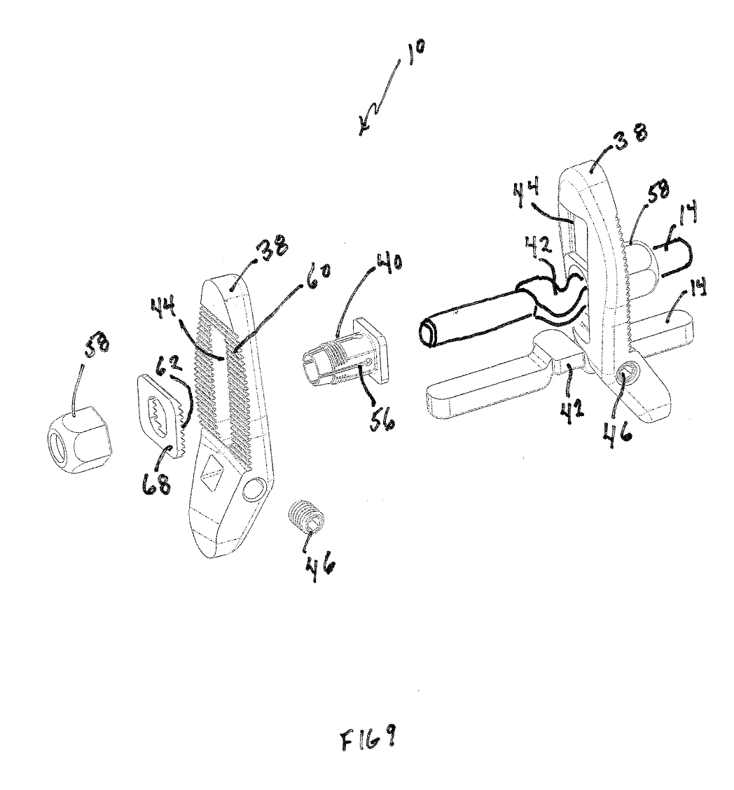

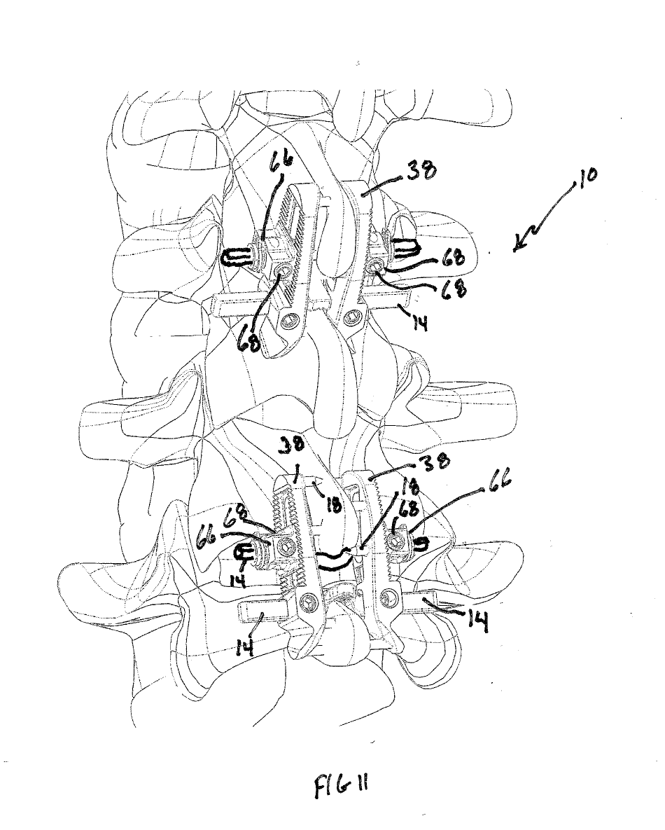

A third exemplary embodiment of the device 10 as shown in FIGS. 8-11 is, in similar fashion to the second exemplary embodiment described above, capable of being adjusted longitudinally so as to set and hold the relative position of two adjacent spinous processes while also permitting adjustment of the transverse members 14 relative to the side plates 38 so as to bring the outlying side plates 38 inward into a secure contact position against the lateral surfaces of the spinous process. In this third exemplary embodiment of the device 10, the transverse members 14 pass through transverse member adjustment slots 44 defined through the side plates 38. Other elements of the device 10, such as the caudal or cephalid directed central sloping areas of the transverse members 14 are also similar to those already described for other embodiments. As shown in FIGS. 8-11 the adjustment of the transverse members 14 upward or downward along the longitudinal axis of the side plates 38 is accomplished by adjustment of the transverse member end portions within the elongated transverse member adjustment slots 44 defined laterally through the body of the side plates 38. As best shown in FIG. 9 when the transverse members 14 are selectively adjusted to the desired location within the adjustment slots 44, their relative positions can be securely locked using the compressive force of a collet-like guide sleeve 40 having circumferentially disposed compression slots 56 that when a locking element such as a threaded end nut 58 is locked down secures the transverse member 14 into position. Additionally, a locking screw can be used to secure the transverse member from unwanted lateral movement. At least a portion of the outward facing lateral surfaces of the side plates 38 can be provided with a textured configuration such as, but not limited to a ratchet surface 60 to support holding the side plates 38 in a locked position relative to the transverse member 14. A complementary opposing textured configuration or ratchet surface 62 can be provided on the locking plate 64. FIG. 11 shows a variation of the third exemplary embodiment of the device 10 as described above in that instead of a threaded end nut 58 as described above, the transverse member 14 is locked into a selected position through the use of a transverse member locking block 66 through which the transverse member 14 passes. A block set screw 68 is used to secure the position of the transverse member 14 within the locking block 66. As shown in FIG. 11, this variation of the third exemplary embodiment also includes the use of surface contact structures such as spikes 18 to facilitate a secure hold on the lateral surfaces of the spinous process.

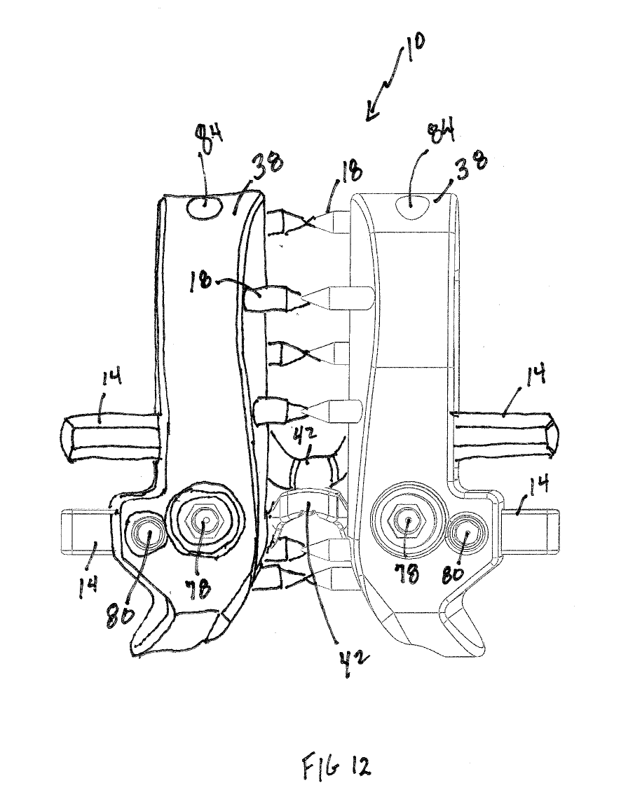

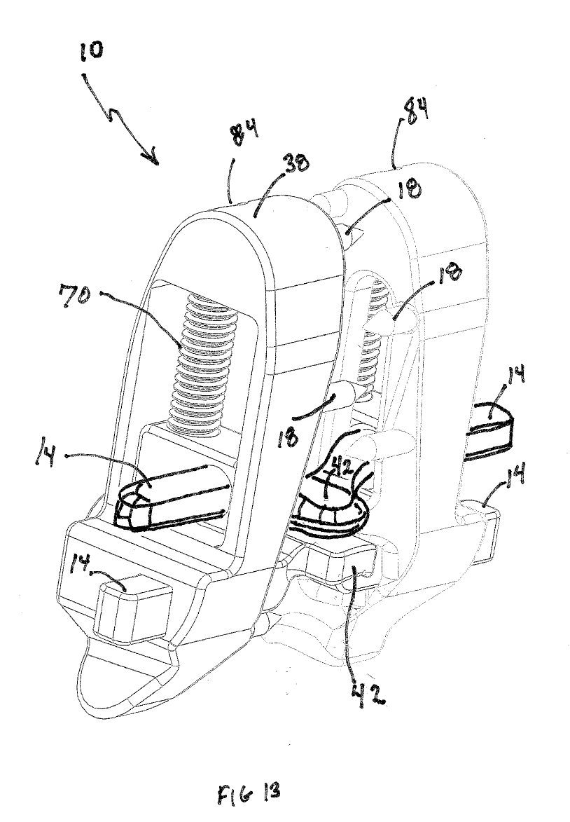

A fourth exemplary embodiment of the device 10 as shown in FIGS. 12-15 also has many similar elements to the earlier described exemplary embodiments that provide the basic function of the device 10, which is to allow adjustment of the device in two dimensions relative to the spine, that is longitudinal and lateral adjustment as described above. As best shown in FIG. 12, the side plates 38 are provided with surface contact structures, such as but not limited to spikes 18, for improved security of the contact to the lateral surface of the spinous process. As shown in FIGS. 13-15, the longitudinal adjustment of the transverse members 14 is accomplished by turning an elongated, vertically orient gear actuator 70 having an integral bevel gear element 72 at its inferior end. A gear drive actuator 74 terminating in a bevel gear complementary to the bevel gear element 72 of the elongated gear 70 when turned interacts with and causes the elongated gear 70 to rotate about its pivot base 86 at the base of the guide/gear box 76 upon which the elongated gear 70 can freely pivot. This interaction, depending upon the direction of rotation causes the guide/gear box to move upward or downward within the side plate 38. In doing so, the side plate 38 carries the transverse member along to the selected position in the side plate 38. It can then be locked into the selected position by the locking screw 78. To accomplish a lateral adjustment of the elements of the device, the lateral plate 38 is moved laterally relative to the slidably connected transverse member such that gripping contact augmented by the surface contact structures such as spikes is achieved, the lateral set screw 80 can be secured into the lateral plate to hold the transverse member 14 in its selected lateral orientation to the spinous process.

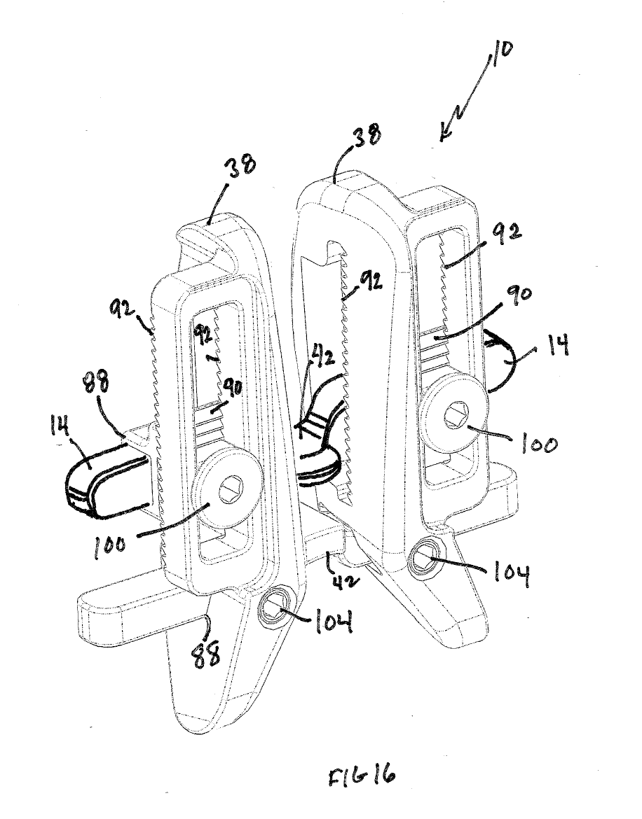

A fifth exemplary embodiment of the device 10 as shown in FIGS. 16-18 is, as other embodiments capable of being adjusted longitudinally so as to set and hold the relative position of two adjacent spinous processes while also permitting adjustment of the transverse members 14 relative to the side plates 38. In this fifth exemplary embodiment of the device 10, the transverse members 14 pass through transverse member carriers 88 having forward facing ratchet teeth 90 or a similarly configured gripping surface that is complementary to rearward facing ratchet teeth 92 along the contact surface of the front wings 94 of the side plates 38. Define through the front wings 94 of the side plates 38 is the carrier access slots 96 through which a carrier locking element 100 can partially extend and make threaded contact with the locking element receptacle 98 on the transverse member carrier 88. When the carrier 88 is adjusted upward or downward along the longitudinal axis of the side plate 38 a selected position can be locked into place by tightening the carrier locking element 100 into the carrier locking element receptacle 98. As the locking element 100 is tightened it forces the forward facing ratchet teeth 90 of the carrier 88 into a locked engagement with the rearward facing ratchet teeth 92 of the front wing of the side plate 94. This locking arrangement is best shown in FIG. 16. Full insertion of the locking element 100 into the locking element receptacle 98 of the transverse member carrier 88 permits locking contact of the distal end 102 of the locking element 110 against the transverse member 14, which adjustable passes laterally through the transverse member carrier portal 102 of the carrier 88. Thus, when the device 10 is selectively positioned in both of the adjustable dimensions, longitudinally and laterally, the locking element 100 can be set to completely secure the device 10. A set screw 104, as with other exemplary embodiments of the device 10 can be used to lock the lower lateral-only adjustment transverse member 14.

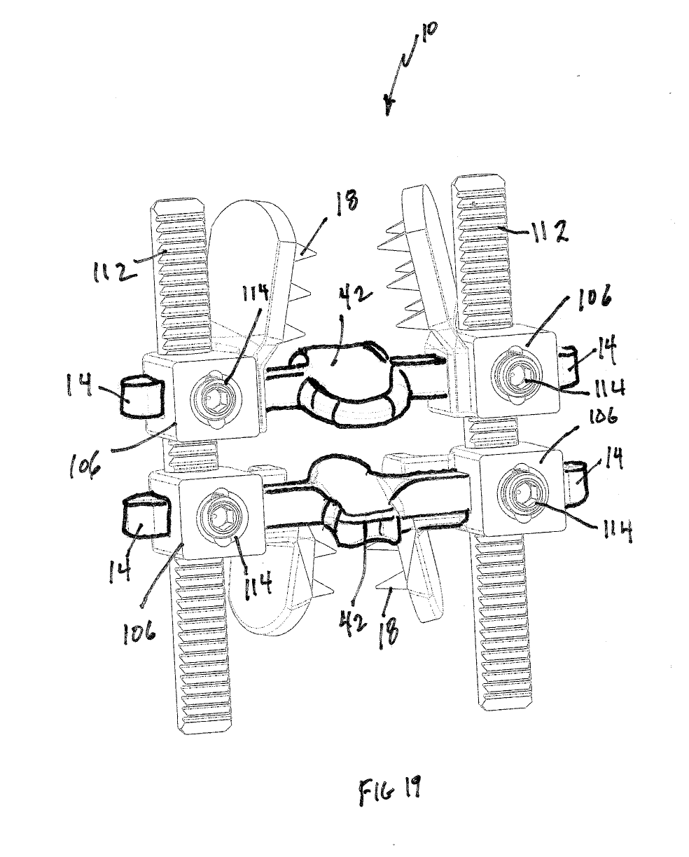

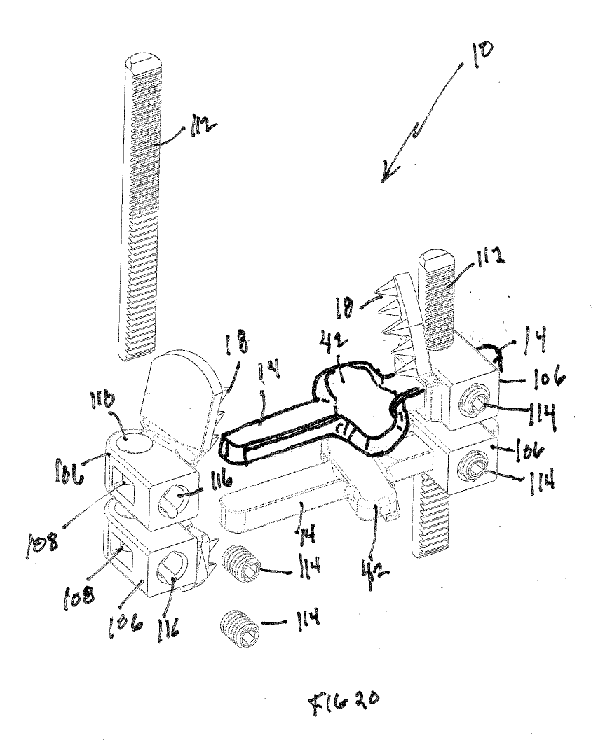

A sixth exemplary embodiment of the device 10 as shown in FIGS. 19-21 is also capable of being adjusted longitudinally and laterally as described for the other exemplary embodiments. In this sixth exemplary embodiment of the device 10, the transverse members 14 pass through transverse member carrier blocks 106, which are configured to allow adjustable slidable passage of the transverse members through transverse member slots 108. Longitudinal elongated legs 112 having forward facing surface contact structures such as ratchet teeth are sized and configured to pass through a vertically oriented elongated leg receiving portals 110, the lumens of which intersect with the lumens of the transverse member slots 108 as shown in FIG. 20. When the longitudinal and lateral slidable adjustment of the transverse members 14 and the elongated legs 112 are selectively positioned, the device set screws 114 can be securely set in the set screw receptacles 116 to fully lock the device 10. Also included in this sixth exemplary embodiment of the device 10 are spinous process engagement wings 116 extending inwardly from the carrier blocks 106. The engagement wings 116 can be provided with surface contact members such as spikes 18 or the like.

The features of the device 10 can be embodied in various configurations and combinations, not all of which have been recited in the examples discussed herein. The components of the device 10 can be manufactured in various sizes of varying relative dimensions and geometry to conform to the natural shape of the spinous process. Furthermore, it is also within the understanding of the inventors that the device can be manufactured of any known materials having the necessary strength and resiliency to meet the operational requirements of the invention. Such variations in the device 10 can be used as necessary to improve the function of the device for the needs of a particular patient.

The device 10 can be manufactured as components by methods known in the art, to include, for example, molding, casting, forming or extruding, and machining processes. The components can be manufactured having a variety of different dimensions so as to provide components that can be selected by the surgeon as being best suited to the anatomical size and conformation of individual patient's vertebral structure. Non-limiting examples of materials that may be used at least in part for components of the device 10 include, for example, implant grade metallic materials, such as titanium, titanium alloy, cobalt- chromium alloys, stainless steel, and the like. Additionally, the structures of the device 10 can be manufactured wholly or in part using non-metallic materials such as, for example, carbon fiber composites, PEEK, PEKK, or other suitable non-metallic materials.

It is also within the concept of the present invention to provide a kit, which can include all components of the inventive device as well as necessary instruments and tools to facilitate the surgical procedure and additional instruments and/or implants or components which can be employed as deemed necessary by the surgeon. Such a kit can be provided with sterile packaging to facilitate opening and immediate use in an operating room.

For each of the exemplary embodiments of the device 10 disclosed herein, the method of use of the device 10 includes providing a bi-dimensionally adjustable spinous process spacer device capable of securely maintaining a selected distance between the spinous processes of two or more adjacent vertebrae and also allowing for lateral positioning of elements of the device 10 so as to make secure contact with the lateral surfaces of the spinous process of the vertebrae to which the device is attached, adjusting the device so as to obtain the desired spacing between the two or more adjacent spinous processes of the vertebrae of interest and selectively adjusting the lateral position of the elongated legs of the device relative to the spinous processes, locking the device securely into the selected position.

Each of the embodiments described above are provided for illustrative purposes only and it is within the concept of the present invention to include modifications and varying configurations without departing from the scope of the invention that is limited only by the claims included herewith.

I claim:

1. A device for use in the treatment of a spinal condition, the device comprising:
a first longitudinal member having a first end and a second end,
a second longitudinal member having a first end and a second end,
at least a first and a second transverse member adjustably connected on a longitudinal and a lateral plane to each of said first and second longitudinal members and holding said first and second members in an approximately parallel orientation one to the other, said first and second transverse members being position between the spinous processes of two adjacent vertebrae and capable of being adjusted longitudinally and locked into position so as to abut against the respective proximate spinous process and hold those adjacent spinous process at a selected distance one from the other and also being capable of being laterally adjusted relative to the longitudinal members so as to enable the longitudinal members to be laterally adjusted toward or away from one another in their approximately parallel orientation,
a position locking mechanism capable of securely holding each of said first and second transverse members in a selected position relative to said first and second longitudinal members and the respective adjacent spinous processes after said first and second transverse members have been selectively adjusted both laterally and longitudinally relative to said longitudinal members and the respective adjacent spinous processes.

2. The device of claim 1, wherein said
at least a first and second transverse process are more than two transverse processes, each of said transverse processes being adjustably connected on said first and second longitudinal members so as to be positioned between adjacent spinous processes of vertebrae.

3. The device of claim 1, wherein said transverse members comprise a first end, a second end, and a central portion connecting said first end to said second end, said central portion being curved in a caudal or cephalic direction along substantially the length of said central portion, said curve being away from the longitudinal axis of said transverse member so as to abut against and hold in position a respective spinous process of an adjacent vertebra.

4. The device of claim 3, wherein said locking mechanism comprises two locking mechanisms, one for each end of said transverse members, each locking mechanism being capable of securely holding its respective end of the transverse member in a selected position relative to said first or second longitudinal member adjustably connected to said transverse members.

5. The device of claim 4, wherein said adjustable connection of said longitudinal members to said transverse members comprises an oblong through portal defined through the first and second ends of the transverse member in a direction generally aligned with the longitudinal axis of the spine, said through portals being configured to permit slidable passage of said longitudinal members through said through portals and to allow both longitudinal and lateral adjustment of said longitudinal members relative to said transverse member,
and said locking mechanism comprises an elongated locking portal defined in the upper surface of said first and second ends of said transverse members so as to permit accesses to the underlying oblong through portal and the longitudinal member adjustably contained therein, a threaded grommet that is sized and configured to be slidably engaged within said elongated locking portal, and a locking screw threadably engaged within said threaded grommet, said locking screw when fully engaged in a locked position in said grommet serving to securely lock said transverse member and said longitudinal member in a selected lateral and longitudinal spatial relationship one to the other.

6. The device of claim 4, wherein said adjustable connection of said longitudinal members to said transverse members comprises an oblong through portal defined through the first and second ends of the transverse member in a direction generally aligned with the longitudinal axis of the spine, said through portals being configured to permit slidable passage of said longitudinal members through said through portals and to allow both longitudinal and lateral adjustment of said longitudinal members relative to said transverse member,
and said locking mechanism comprises a clip engageable with each end of said transverse members, said clip having an outward bias that when inserted into said oblong through portal is capable of exerting a bias against said longitudinal member slidably positioned in said through portal, said clip comprising a locking notch that catches an edge of said through portal so as to lock the clip into a holding position within said through portal and securely hold said longitudinal member in a selected lateral and longitudinal spatial relationship one to the other.

7. The device of claim 1, wherein said longitudinal members are provided as side plates in a substantially parallel orientation one to the other, said side plates each defining an upper and lower transverse member adjustment slot, said upper transverse member adjustment slots being elongated to permit both longitudinal adjustment and lateral adjustment of said transverse member relative to said side plates, said first and second transverse members each having a first end and a second end connected by a central portion, said first end and said second end of said transverse member being slidably engaged within said slots of said side plates, said transverse members being held in a longitudinal relationship to said upper slots in said side plates by a collet lock mechanism.

8. The device of claim 1, wherein said longitudinal members are provided as side plates in a substantially parallel orientation one to the other, said side plates each defining an upper and lower transverse member adjustment slot, said upper transverse member adjustment slots being elongated to permit both longitudinal adjustment and lateral adjustment of said transverse member relative to said side plates, said first and second transverse members each having a first end and a second end connected by a central portion, said first end and said second end of said transverse member being slidably engaged within said slots of said side plates;
a textured configuration provided on the outward facing lateral surfaces of said side plates 38;
a locking plate configured to allow the each end of said transverse member to slidably pass there through comprising an inwardly surface having a textured configuration complementary to and capable of lockably engaging with said textured configuration of said outward facing lateral surfaces of said side plates,
a locking element capable of securing said locking plate firmly against said outward surface of said side plate,
wherein when said locking elements are secured in a locked position, said side plates are firmly locked into a longitudinal and lateral plane spatial relationship with said transverse members.

9. The device of claim 8, wherein said textured configuration is a ratched surface.

10. The device of claim 1, wherein said longitudinal members are provided as side plates in a substantially parallel orientation one to the other, each of said side plates defining an upper and lower transverse member adjustment slot, said upper transverse member adjustment slots being elongated to permit both longitudinal adjustment and lateral adjustment of said transverse member relative to said side plates, said first and second transverse members each having a first end and a second end connected by a central portion;
an elongated, vertically orient gear actuator rotatably housed within said upper transverse member adjustment slots, said elongated gear actuator,
a gear box moveably connected to said elongated gear actuator and slidably connected to said transverse member,
wherein rotational movement of said elongated gear actuator causes movement of said gear box and said slidably connected transverse member.

11. The device of claim 10, wherein said textured configuration is a ratched surface.

12. The device of claim 1, wherein said longitudinal members are provided as side plates in a substantially parallel orientation one to the other, each of said side plates defining an upper and lower transverse member adjustment slot, said upper transverse member adjustment slots being elongated to permit both longitudinal adjustment and lateral adjustment of said transverse member relative to said side plates, said first and second transverse members each having a first end and a second end connected by a central portion;
a transverse member carrier configured slidably connect to the ends of said upper transverse member and provided with a forward facing textured contact surface; said transverse members 14 configured to slidably pass through said transverse member carriers, said carriers comprising a forward facing textured surface;
front wings of said side plates configured in a slidable relationship to said carriers, said front wings comprising a rearward facing textured surface complementary to said textured surface of said slidably engaged carriers,
wherein when said transverse member is selectively adjusted in the longitudinal and lateral planes of the device relative to the side plates, said complementary textured surfaces of said carriers and said front wings are capable of being lockably engaged so as to hold said transverse members and said side plates in a locked longitudinal and lateral plane relationship one to the other.

13. The device of claim 12, wherein said textured configuration is a ratched surface.

14. the transverse members 14 pass through transverse member carrier blocks 106, which are configured to allow adjustable slidable passage of the transverse members through transverse member slots 108. Longitudinal elongated legs 112 having forward facing surface contact structures such as ratchet teeth are sized and configured to pass through a vertically oriented elongated leg receiving portals 110, the lumens of which intersect with the lumens of the transverse member slots 108 as shown in FIG. 20. When the longitudinal and lateral slidable adjustment of the transverse members 14 and the elongated legs 112 are selectively positioned, the device set screws 114 can be securely set in the set screw receptacles 116 to fully lock the device 10. Also included in this sixth exemplary embodiment of the device 10 are spinous process engagement wings 116 extending inwardly from the carrier blocks 106. The engagement wings 116 can be provided with surface contact members such as spikes 18 or the like.

15. The device of claim 1, wherein said longitudinal members are configured as elongated legs, said elongated legs having a textured configuration on the forward facing surface.

16. The device of claim 15, wherein said textured configuration is a ratched surface.

17. A method of treating a subject for a spinal disorder, the method comprising:
providing the device of claim 1;
surgically accessing the area of interest on the spinal column of a subject;
applying said device to the affected vertebra.
